SEQUENCE LISTING (6 sequences)

SEQ ID NO: 1 - DNA, 19 bp, Artificial Sequence
  oligonucleotide primer sequence GeneN-B-F3
accgaagagctaccagacg

SEQ ID NO: 2 - DNA, 20 bp, Artificial Sequence
  oligonucleotide primer sequence GeneN-B-B3
tgcagcattgttagcaggat

SEQ ID NO: 3 - DNA, 40 bp, Artificial Sequence
  oligonucleotide primer sequence GeneN-B-FIP
tctggcccagttcctaggtagttcgtggtggtgacggtaa

SEQ ID NO: 4 - DNA, 40 bp, Artificial Sequence
  oligonucleotide primer sequence GeneN-B-BIP
agacggcatcatatgggttgcacgggtgccaatgtgatct

SEQ ID NO: 5 - DNA, 25 bp, Artificial Sequence
  oligonucleotide primer sequence GeneN-B-LF
ccatcttggactgagatctttcatt

SEQ ID NO: 6 - DNA, 21 bp, Artificial Sequence
  oligonucleotide primer sequence GeneN-B-LB
actgagggagccttgaataca

(19) United States
(12) Patent Application Publication
(10) Pub. No.: US 2023/0158489 A1
(43) Pub. Date: May 25, 2023

(54) OPTICAL DETECTION SYSTEM

(71) Applicant: Danmarks Tekniske Universitet, Kongens Lyngby (DK)

(72) Inventors: Dang Duong BANG, Kongens Lyngby (DK); Anders WOLFF, Kongens Lyngby (DK); Sune Zoëga ANDREASEN, Kongens Lyngby (DK); Trieu NGUYEN, Kongens Lyngby (DK); Van Ncog HUYNH, Kongens Lyngby (DK); Nguyen Thi Thu TRANG, Kongens Lyngby (DK); Quyen Than LINH, Kongens Lyngby (DK); Mohsen GOLABI, Kongens Lyngby (DK); Tien Ngo ANH, Kongens Lyngby (DK); Vinayaka Aaydha CHIDAMBARA, Kongens Lyngby (DK)

(21) Appl. No.: 17/995,392

(22) PCT Filed: Mar. 31, 2021
(86) PCT No.: PCT/DK2021/050095

§ 371 Date: Oct. 3, 2022

(30) Foreign Application Priority Data
Apr. 5, 2020 (DK) PA202070211
May 7, 2020 (EP) 20173505.7

(51) Int. Cl. B01L 3/00; G01N 21/03; G01N 21/64

(52) U.S. Cl. CPC B01L 3/5027; G01N 21/0303; G01N 21/6452; B01L 3/50851; B01L 3/50853

(57) ABSTRACT

The present invention relates to a lab-on-a-chip (LOAC)-system for the rapid detection of e.g. pathogens. The system comprises a tabletop detection apparatus and a portable optical detection cartridge for being received in the inner of the detection apparatus, the cartridges comprising a plurality of test wells for detecting a desired chemical reaction taking place within a respective test well. In embodiments of the invention, the optical detection cartridge is pre-loaded with suitable respective reagents selective for a disease pathogen such as severe acute respiratory syndrome coronavirus 2 (SARS-CoV-2).

The invention relates to the field of lab-on-a-chip, an optical detection system and tests for diseases detectable therewith.

### BACKGROUND

The detection of pathogens is key to the prevention and identification of problems related to health and safety. Legislation is particularly tough in areas such as the food industry, where failure to detect an infection may have terrible consequences. It may further be crucial for the outcome of a patient with an infection that the type of pathogen causing the infection is diagnosed in an early stage or prior to starting a treatment of the patient.

In spite of the real need for obtaining analytical results in the shortest time possible, traditional and standard detection methods may take up to 7 or 8 days to yield an answer or may require multiple and time-consuming steps from the technician involved, wherein there is a risk of introducing an error in each step of the process.

This is clearly insufficient, and many researchers have recently geared their efforts towards the development of rapid methods which reduces the amount of work required by the technician involved, and which greatly reduces the time span from test sampling to test result.

Hence, it is an object of the present invention to provide a solution to the above problems and provide a system, which is able provide a result in as few steps as possible from taking the test sample to final result. It is further an object of the present invention to provide a system, which provides a final analytical result to the pathogen test within a reasonable timeframe.

### SUMMARY

The present invention relates to a lab-on-a-chip (LOAC)-system for the rapid detection of e.g. pathogens. The system comprises a tabletop detection apparatus, e.g. an optical detection apparatus, and a portable optical detection cartridge for being received in the inner of the detection apparatus, the cartridges comprising a plurality of test wells for detecting a desired chemical reaction taking place within a respective test well, when at least one chemical reaction within the respective test well results in a reaction by-product which is detectable by optical means such as by absorbance of light.

Disclosed herein in a first aspect is a portable optical detection cartridge configured for being received in an optical detection apparatus, wherein the portable optical detection cartridge comprises: alid; a holder; and a chip; wherein the chip at least comprises: a plurality of test wells, wherein a number of the plurality of test wells are pre-loaded with suitable reagents allowing a loop-mediated isothermal amplification (LAMP) between a test sample and the reagents to take place within each test well when the test sample is administered to the test well; and a plurality of optical structures, wherein each optical structure is configured for reflecting light in a 90 degree angle in at least three reflection directions; wherein at least one of the optical structures is configured for receiving light from the optical detection apparatus and for transmitting the light to the test sample; and wherein two optical structures are positioned on opposite sides of each test well.

The cartridge is a part of an optical detection system, where the cartridge can be easily removed and replaced when it has been used for a test in an optical detection apparatus.

By optical detection is meant detection by e.g. fluorescence emitting from a sample or absorption of light through the sample.

A test well is a well comprising all the reagents for running a loop-mediated isothermal amplification. In one or more examples, all that is needed to be added to the well may therefore be the test sample or a control sample, hereby obtaining a sample mixture in the test well.

Loop-mediated isothermal amplification (LAMP) is a technique for the amplification of RNA or DNA. LAMP has major advantages such as simplicity, ruggedness, and low cost. LAMP can be used as a simple screening assay in the field or at the point of care by clinicians. Because LAMP is isothermal, which eradicates the need for expensive thermocyclers used in conventional PCR, it may be a particularly useful method for infectious disease diagnosis in low and middle income countries.

Further, LAMP has also been observed to be less sensitive (more resistant) than PCR to inhibitors in complex samples such as blood, likely due to use of a different DNA polymerase (typically Bst—Bacillus stearothermophilus—DNA polymerase rather than Taq polymerase as in PCR). This feature of LAMP may be useful in low-resource or field settings where a conventional DNA or RNA extraction prior to diagnostic testing may be impractical.

In contrast to the polymerase chain reaction (PCR) technology, in which the reaction is carried out with a series of alternating temperature steps or cycles, LAMP is an isothermal nucleic acid amplification technique carried out at a constant temperature. The LAMP technique does not require a thermal cycler.

In LAMP, the target sequence is normally amplified at a constant temperature of 60-65° C. using either two or three sets of primers and a polymerase with high strand displacement activity in addition to a replication activity. For example, four different primers may be used to amplify six distinct regions on the target gene, which increases specificity. An additional pair of “loop primers” may further accelerate the reaction. The amount of DNA produced in LAMP is normally considerably higher than PCR-based amplification.

The amplification product can be detected via photometry, wherein a reaction e.g. may be followed by detection of fluorescence using e.g. intercalating dyes. Dyes may be used to create a visible color change that can be measured by instrumentation. Dye molecules intercalate or directly label of the RNA or DNA, may in turn be correlated with the number of copies initially present.

By reflection directions is meant the direction of which the light emitted onto the optical structure is reflected around the optical structure. This means that e.g. if light is emitted onto the optical structure from above it may be reflected in a 90 degree angle in three directions with 120 degrees between them, or it may be reflected in a 90 degree angle in four directions with 90 degrees between them.

A test sample is the sample in which one is interested in knowing if it comprises a disease pathogen. A test sample could e.g. be a blood or saliva sample from a patient, it could be a water sample from a water drilling, or it could be a swipe from a surface on a hospital or in a slaughterhouse. The test sample is tested to see if it comprises a disease pathogen. A disease pathogen is a virus, bacteria, fungi, or parasite.

Viruses are small particles, typically between 20 and 300 nanometers in length. Viruses require a host cell to replicate. Some of the diseases that are caused by viral pathogens include smallpox, influenza, mumps, measles, chickenpox, ebola, HIV, and rubella. Pathogenic viruses are often mainly from the families: Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae.

Pathogenic bacteria can cause infectious diseases. Pathogenic bacteria have several ways that they can cause disease. They can either directly affect the cells of their host, produce endotoxins that damage the cells of their host, or cause a strong enough immune response that the host cells are damaged. One of the bacterial diseases with the highest disease burden is tuberculosis, caused by the bacterium Mycobacterium tuberculosis. Pathogenic bacteria contribute to other globally significant diseases, such as pneumonia, which can be caused by bacteria such as Streptococcus and Pseudomonas, and foodborne illnesses, which can be caused by bacteria such as Shigella, Campylobacter, and Salmonella. Pathogenic bacteria also cause infections such as tetanus, typhoid fever, diphtheria, syphilis, and leprosy.

Fungi are eukaryotic organisms that can function as pathogens. There are several known fungi that are pathogenic to humans, including Candida albicans, which is the most common cause of thrush, and Cryptococcus neoformans, which can cause a severe form of meningitis.

Some eukaryotic organisms, including a number of protozoa and helminths, are human parasites.

By pre-loaded with suitable reagents is meant that when the test sample is applied to the test well, the well already comprises the suitable reagents.

Disclosed herein in a second aspect is a lab-on-a-chip system comprising a portable optical detection cartridge, wherein the portable optical detection cartridge comprises a plurality of test wells, wherein a number of the plurality of test wells are pre-loaded with suitable reagents allowing a loop-mediated isothermal amplification (LAMP) between a test sample and the reagents to take place within each test well when the test sample is administered to the test well, and a plurality of optical structures, wherein each optical structure is configured for reflecting light in at least three reflection directions; and wherein the lab-on-a-chip system further comprises an optical detection apparatus configured for receiving the portable optical detection cartridge, wherein the portable optical detection cartridge comprises at least a first optical structure and a second optical structure, and wherein the optical detection apparatus comprises at least one light source for illuminating the first optical structure and at least one light detector for receiving light transmitted from the second optical structure.

Disclosed herein in a third aspect is the use of a portable optical detection cartridge configured for being received in an optical detection apparatus, wherein the portable optical detection cartridge comprises a plurality of test wells, wherein a number of the plurality of test wells are pre-loaded with suitable reagents allowing a loop-mediated isothermal amplification (LAMP) between a test sample and the reagents to take place within each test well when the test sample is administered to the test well; and a plurality of optical structures, wherein each optical structure is configured for reflecting light in at least three reflection directions or a lab-on-a-chip system comprising a portable optical detection cartridge, wherein the portable optical detection cartridge comprises a plurality of test wells, wherein a number of the plurality of test wells are pre-loaded with suitable reagents allowing a loop-mediated isothermal amplification (LAMP) between a test sample and the reagents to take place within each test well when the test sample is administered to the test well, and a plurality of optical structures, wherein each optical structure is configured for reflecting light in at least three reflection directions; and wherein the lab-on-a-chip system further comprises an optical detection apparatus configured for receiving the portable optical detection cartridge, wherein the portable optical detection cartridge comprises at least a first optical structure and a second optical structure, and wherein the optical detection apparatus comprises at least one light source for illuminating the first optical structure and at least one light detector for receiving light transmitted from the second optical structure for qualitative or quantitative detection of a disease pathogen in a test sample.

Disclosed herein in a fourth aspect is a method of detecting a presence of a disease pathogen in a test sample, wherein the method comprises the steps of:
Adding the test sample to at least one test well in a portable optical detection cartridge configured for being received in an optical detection apparatus, wherein the portable optical detection cartridge comprises a plurality of test wells, wherein a number of the plurality of test wells are pre-loaded with suitable reagents allowing a loop-mediated isothermal amplification (LAMP) between a test sample and the reagents to take place within each test well when the test sample is administered to the test well; and a plurality of optical structures, wherein each optical structure is configured for reflecting light in at least three reflection directions;
Inserting the portable optical detection cartridge into a optical detection apparatus;
Heating the portable optical detection cartridge within the optical detection apparatus to a predetermined temperature, preferably between 60° C. and 70° C.;
Maintaining the predetermined temperature until the method of detecting the disease pathogen in the test sample is complete;
Leaving the loop-mediated isothermal amplification between the test sample and the reagents to take place within the test well for a predetermined amount of time;
Emitting light from a light source in the optical detection apparatus to at least one of the optical structures hereby emitting light to the at least one test well in the portable optical detection cartridge;
Detecting light with a photo detector in the optical detection apparatus,
wherein the light is emitted from the at least one test well in the portable optical detection cartridge to one of the optical structures reflecting light to the photodetector, hereby detecting the presence of the disease pathogen in the test sample.

Any of the below embodiments may be combined with any of the above aspects.

In one or more embodiments, the temperature in the test wells is elevated to a predetermined temperature after the test sample is administered to the test well.

In one or more embodiments, each optical structure is configured for reflecting light in four reflection directions.

In one or more embodiments, each optical structure is shaped as a pyramid having four reflecting surfaces.

In one or more embodiments, each optical structure is shaped as a pyramid having four reflecting surfaces and each optical structure is configured for reflecting light in four reflection directions.

In one or more embodiments, each optical structure is a total internal reflection structure.

By total internal reflection (TIR) is meant an optical phenomenon in which a surface reflects the light in the same manner as like a mirror with no loss of brightness. In general, TIR occurs when waves in one medium reach the boundary with another medium at a sufficiently slanting angle, provided that the second (“external”) medium is transparent to the waves and allows them to travel faster than in the first (“internal”) medium.

In one or more embodiments, each optical structure is configured for receiving light from the optical detection apparatus or for transmitting light from another optical structure.

In one or more embodiments, each optical structure is configured for receiving light from the optical detection apparatus and for transmitting light from another optical structure.

In one or more embodiments, each optical structure is configured for receiving light emitted from the test sample to the optical detection apparatus.

In one or more embodiments, at least one of the optical structures is configured for receiving light from the optical detection apparatus.

In one or more embodiments, at least one of the optical structures is configured for transmitting light from another optical structure.

In one or more embodiments, each optical structure is configured for receiving light from the optical detection apparatus or configured for transmitting light from another optical structure and light emitted from the test sample to the optical detection apparatus.

In one or more embodiments, at least one of the optical structures is configured for receiving light from the optical detection apparatus and for transmitting the light to the test sample and at least one other of the optical structures is configured to transmit light emitted from the test sample or passing through the test sample to the optical detection apparatus.

In one or more embodiments, the portable optical detecting cartridge is configured for being received in an optical detection apparatus comprises six light sources and five light detectors and wherein the portable optical detection cartridge comprises eleven optical structures, wherein each of the optical structures are configured for either reflecting light from one of the six light sources or reflecting light to one of the five light detectors when the portable optical detection cartridge is inserted into the optical detection apparatus.

In one or more embodiments, along at least two of the reflection directions, defined by each optical structure, another optical structure is positioned and wherein two optical structures are positioned on opposite sides of each test well.

In one or more embodiments, the suitable reagents are gelified and the suitable reagents form a sample mixture when the test sample is added, such that the sample mixture is configured for emitting fluorescence light when illuminated with light from the optical detection apparatus, wherein a first of the plurality of optical structures are configured for reflecting the light from the optical detection apparatus through a test well comprising the sample mixture and at least a second of the plurality of optical structures are configured for reflecting the emitted fluorescence to the optical detection apparatus.

Gelified reagents may be used to ensure that the reagents remain inside the test well during handling prior to adding the test sample to the test wells.

In one or more embodiments, the optical detection apparatus comprises six light sources and five light detectors and wherein the portable optical detection cartridge comprises eleven optical structures, wherein each of the optical structures are configured for reflecting light from one of the six light sources or to one of the five light detectors when the portable optical detection cartridge is inserted into the optical detection apparatus.

In one or more embodiments, the plurality of test wells is twelve test wells, and where the plurality of optical structures is eleven optical structures.

In one or more embodiments, along at least two of the reflection directions, defined by each optical structure, another optical structure is positioned.

In one or more embodiments, at least one of the optical structures is configured for reflecting light to four of the other optical structures.

In one or more embodiments, at least one of the optical structures is configured for reflecting light from four of the other optical structures.

In one or more embodiments, at least one of the optical structures is configured for reflecting light to and from four of the other optical structures.

In one or more embodiments, each optical structure is configured for reflecting light at a reflection angle between 45-135 degrees, such as between 60-120 degrees, such as 75-105 degrees, such as 85-95 degrees, or such as approximately 90 degrees.

In one or more embodiments, two optical structures are positioned on opposite sides of each test well.

In one or more embodiments, each optical structure is configured for reflecting light in five, six, seven, eight, or more reflection directions.

In one or more embodiments, the portable optical detection cartridge comprises a chip comprising the plurality of test wells and the plurality of optical structures, a holder for holding the chip, and a lid for closing access to the test wells after sample has been supplied to one or more of the test wells. In one or more embodiments, the chip is an injection molded chip. In one or more embodiments, the holder is fabricated from a black polymeric material, and the lid is made from polymerase chain reaction (PCR) tape.

In one or more embodiments, the portable optical detection cartridge further comprises a temporary seal for closing access to the test wells, wherein the temporary seal is removed before test sample is supplied to one or more of the test wells.

In one or more embodiments, the plurality of test wells are absent of fluidic-connection channels connecting the test wells.

By absent of fluidic-connection is meant that each test well is a standalone well, meaning that after the addition of a test sample to a test well, the content/sample mixture in the test well is not transferred to any other test well, but the whole detection and LAMP is a one-pot one-step procedure.

In one or more embodiments, the plurality of test wells comprises at least two control test wells including at least one negative control test well and at least one positive control test well.

In one or more embodiments, the suitable reagents are gelified to ensure that they remain inside the test well during handling prior to supplying a test sample to the plurality of test wells.

In one or more embodiments, the sample and the suitable reagents form a sample mixture configured for emitting fluorescence light when illuminated with light from the optical detection apparatus, wherein a first of the plurality of optical structures are configured for reflecting the light from the optical detection apparatus through a test well with the sample mixture and at least a second of the plurality of optical structures are configured for reflecting the emitted fluorescence to the optical detection apparatus.

In one or more embodiments, the plurality of test wells are pre-loaded with reagents selective for a disease pathogen, such as adenoviruses, herpesviruses, poxviruses, parvoviruses, reoviruses, coronaviruses, picornaviruses, togaviruses, orthomyxoviruses, rhabdoviruses, retroviruses, or hepadnaviruses.

In one or more embodiments, the plurality of test wells are pre-loaded with reagents selective for a disease pathogen such as severe acute respiratory syndrome coronavirus 2 (SARS-CoV-2).

In one or more embodiments, the optical detection cartridge is pre-loaded with suitable respective reagents selective for a disease pathogen such as severe acute respiratory syndrome coronavirus 2 (SARS-CoV-2).

In one or more embodiments, the optical detection apparatus comprises a plurality of light sources and a plurality of light detectors. In one or more embodiments, the light source(s) are light emitting diodes (LEDs).

In one or more embodiments, the optical detection apparatus comprises a plurality of light sources and a plurality of light detectors and wherein the light sources are light emitting diodes (LEDs).

In one or more embodiments, the optical detection apparatus is configured for performing a detection cycle comprising a sequence of illumination-detection steps, wherein in each illumination-detection step:
a light source illuminates a first optical structure;
one or more test wells are illuminated by the light reflected by the optical structure through the one or more test wells; and
one or more second optical structures reflect light emitted from the sample in the test well and/or the light transmitted through the test well to one or more light detectors in the optical detection apparatus.

In one or more embodiments, only one light source is turned on in each illumination-detection step.

In one or more embodiments, each light detector is configured for detecting light transmitted through at least two test wells.

In one or more embodiments, each light detector is configured for detecting light emitted from at least two test wells.

In one or more embodiments, each light detector is configured for detecting light transmitted through and/or emitted from at least two test wells.

In one or more embodiments, the optical detection apparatus comprises six light sources and five light detectors and wherein the portable optical detection cartridge comprises eleven optical structures each reflecting light from one of the six light sources or to one of the five light detectors.

In one or more embodiments, the detection cycle comprises six illumination-detection steps.

In one or more embodiments, the optical detection apparatus comprises one or more heating sources for heating the test wells.

In one or more embodiments, the optical detection apparatus is a loop-mediated isothermal amplification (LAMP) optical detection apparatus.

In one or more embodiments, the heating source(s) are configured for maintaining the temperature approximately constant during detection.

In one or more embodiments, the optical detection apparatus comprises six light sources and five light detectors, wherein the portable optical detection cartridge comprises eleven optical structures, wherein each of the optical structures are configured for either reflecting light from one of the six light sources or reflecting light to one of the five light detectors, wherein the detection cycle comprises six illumination-detection steps each step turning on a different of the six light sources, and wherein the optical detection apparatus comprises one or more heating sources for heating the test wells, wherein the one or more heating sources are configured for maintaining an approximately constant temperature during the loop-mediated isothermal amplification between the test sample and the reagents and for maintaining an approximately constant temperature during detection.

### BRIEF DESCRIPTION OF THE DRAWINGS

Various examples are described hereinafter with reference to the figures. Like reference numerals refer to like elements throughout. Like elements will, thus, not be described in detail with respect to the description of each figure. It should also be noted that the figures are only intended to facilitate the description of the examples. They are not intended as an exhaustive description of the claimed invention or as a limitation on the scope of the claimed invention. In addition, an illustrated example needs not have all the aspects or advantages shown. An aspect or an advantage described in conjunction with a particular example is not necessarily limited to that example and can be practiced in any other examples even if not so illustrated, or if not so explicitly described.

FIGS. 1A-B shows an injection molded LOAC chip (FIG. 1A) and a fully assembled cartridge comprising the holder and lid (FIG. 1B).

FIG. 2 shows an exemplary user interface after running a Loop-Mediated Isothermal Amplification (LAMP) test.

FIG. 3 shows that the LOAC system may both work as a stand-alone instrument and with a PC based application.

FIGS. 4A-C show a protocol for loading and sealing the LOAC cartridge for running LAMP.

FIGS. 5A-B show a detection principle used in the LOAC system with FIG. 5A being a top-down view of the LOAC chip receiving and reflecting light and FIG. 5B being a side-view of the light source and photo detector in the optical detection system and the LOAC chip.

FIG. 6 shows a detection cycle, wherein by turning on and off the light sources and paired photodetectors sequentially.

FIG. 7 shows an on-cartridge test of one-step RT-LAMP (spiked inactivated AIV in oral swap samples).

FIG. 8 shows an on-tube testing the sensitivity of one-step RT-AIV-LAMP kit.

FIG. 9 shows a detection of AIV using the LOAC system and cartridge to detect inactivated AIV with different subtypes.

FIG. 10 shows a flowchart for stand-alone use of the LOAC system.

FIG. 11 shows an exemplary part for assembly of LOAC cartridge.

### DETAILED DESCRIPTION

Exemplary examples will now be described more fully hereinafter with reference to the accompanying drawings. In this regard, the present examples may have different forms and should not be construed as being limited to the descriptions set forth herein. Accordingly, the examples are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The spatially relative terms “bottom”, “top”, “below”, “beneath”, “less”, and the like, may be used herein for ease of description to describe the relationship between one element or component and another element or component as illustrated in the drawings. It will be understood that the spatially relative terms are intended to encompass different orientations of the device in use or operation, in addition to the orientation depicted in the drawings. For example, in the case where a device illustrated in the drawings is turned over, elements described as being “below” or “beneath” another element would then be oriented “above” another element. Accordingly, the illustrative term “below” or “beneath” may include both the “lower” and “upper” orientation positions, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below, and thus the spatially relative terms may be interpreted differently depending on the orientations described.

Throughout the specification, when an element is referred to as being “connected” to another element, the element is “directly connected” to the other element, or “electrically connected” to the other element with one or more intervening elements interposed therebetween.

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

Exemplary examples are described herein with reference to cross section illustrations that are schematic illustrations of idealized examples, wherein like reference numerals refer to like elements throughout the specification. As such, variations from the shapes of the illustrations as a result, for example, of manufacturing techniques and/or tolerances, are to be expected. Thus, examples described herein should not be construed as limited to the particular shapes of regions as illustrated herein but are to include deviations in shapes that result, for example, from manufacturing. For example, a region illustrated or described as flat may have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims. Some of the parts which are not associated with the description may not be provided in order to specifically describe exemplary examples of the present disclosure.

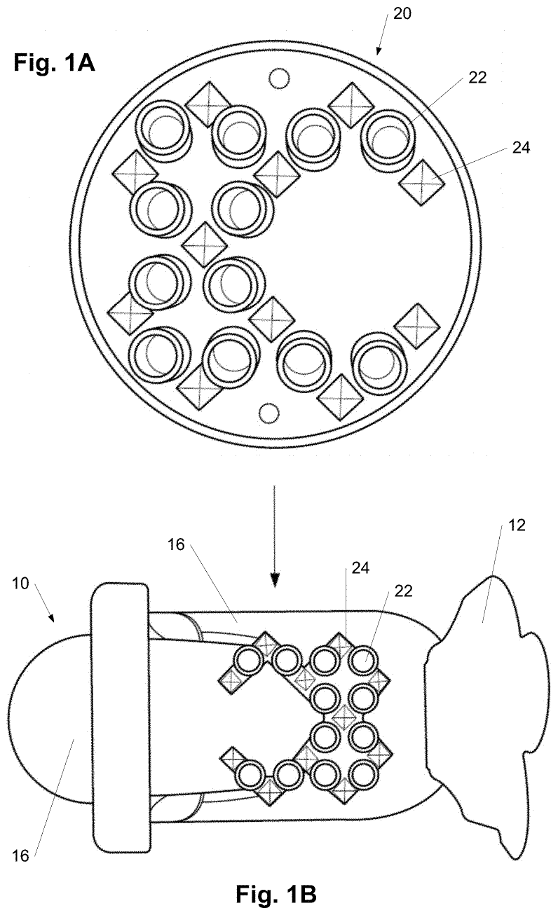

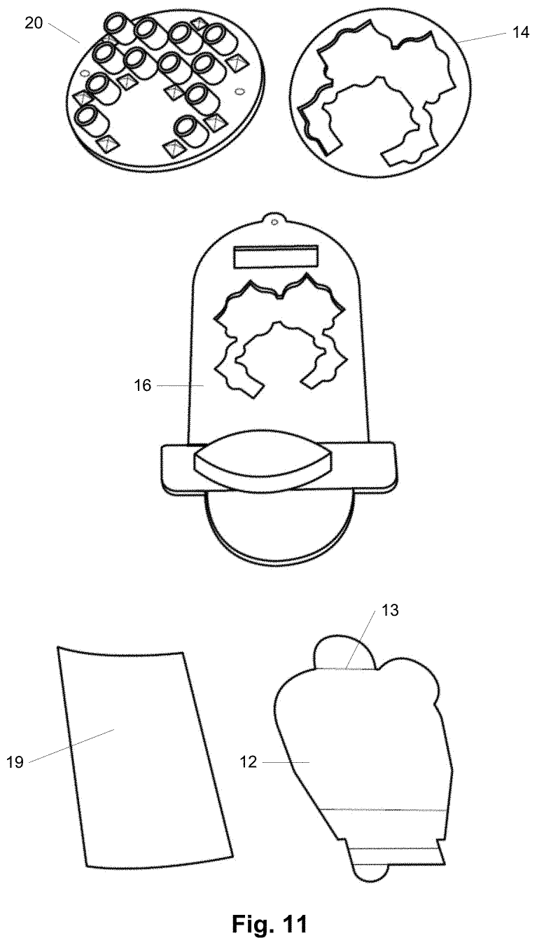

A lab-on-a-chip (LOAC) portable optical detection cartridge 10 is shown in in FIG. 1B. The LOAC cartridge 10 comprises three parts as shown in FIG. 11; a chip 20, a holder 16, and a lip 12. The chip 20 as shown in FIG. 1A may be made an injection molded chip (Topas, grade 5013L-10). The holder 16 may be fabricated from black PMMA (3 mm thick, laser cut). The lid 12 may be made from PCR tape (BioRad “Microseal B”). The lid 12 is preferably integrated with the holder 16. The lid 12 is normally designed to include release liners, for easy handling, even when wearing gloves. This three-part design of the cartridge 10 assures that a user will never get in direct contact with heaters (up to 85° C.) in the detection apparatus 30. Further, the non-symmetric design of the cartridge holder 16 is automatically aligned with sensor arrays in the apparatus 30 (possibly aided by the ball-spring plungers). Also, the lid 12 is integrated and attached to the holder 16, in order to make a single, easy-to-use, unit with no separate handling of lids, seals, etc.

The LOAC cartridge 10 may also be assembled from five components as shown in FIG. 11: A chip 20, a piece of tape 14, a holder 16, a temporary seal 19, and a lid 12. The tape 14 may be a piece of laser cut PSA (double adhesive tape). The holder may be a laser cut holder in black PMMA. The temporary seal 19 may be ¼ of a pressure adhesive PCR tape. The lid 12 may be laser cut from another piece of PCR tape.

The chip 20 comprises a plurality of test wells 22 and optical structures 24 as shown in FIG. 1A. The optical structures 24 may be total internal reflection (TIF) pyramids for facilitating the optical detection. The pyramid structure ensures that when light illuminates one or more of the sides of the pyramid, the light is reflected at an angle of approximately 90 degrees.

The LOAC cartridges 10 are preferably single-use, and comes preloaded with chemicals necessary for running the tests. Normally, the test wells 22 are pre-loaded with suitable reagents allowing a loop-mediated isothermal amplification (LAMP) between a test sample and the reagents to take place within each test well 22 when the test sample is administered to the test well 22.

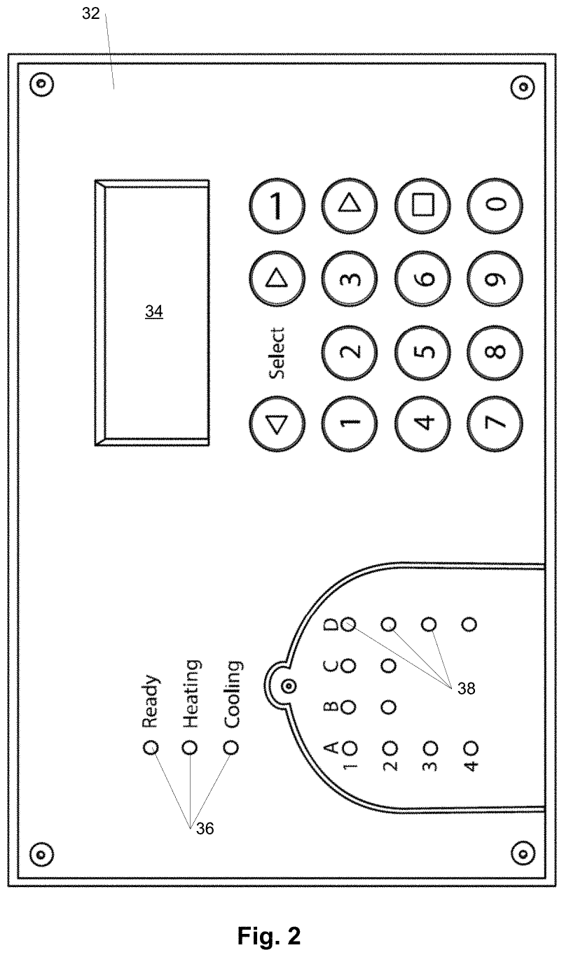

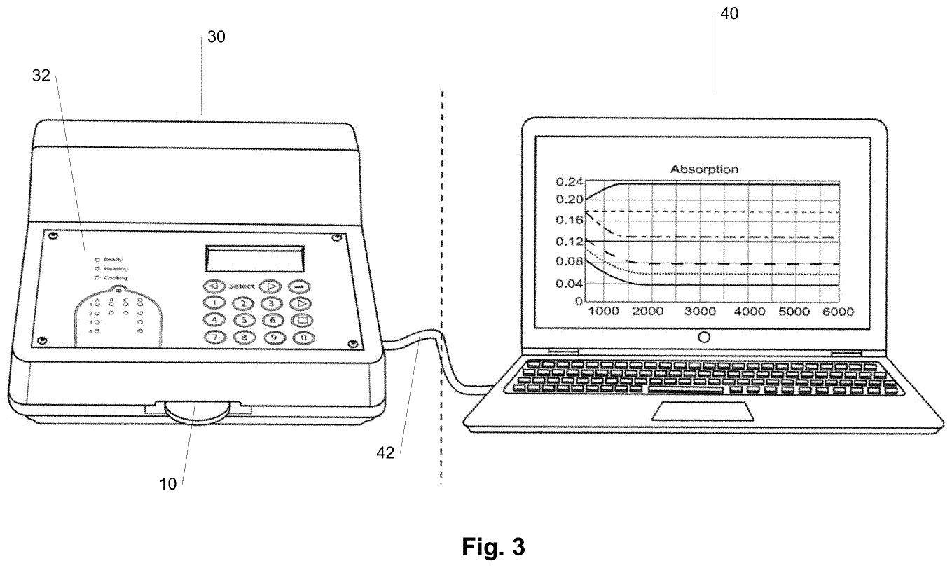

The lab on a chip (LOAC) detection system comprises both mechanical as well as electronic parts and components. All components are preferably packaged in a tabletop instrument 30 enclosure as e.g. shown in FIG. 3. The top of the instrument normally comprises a user interface 32 as shown in FIG. 2. The instrument 30 is normally an optical detection apparatus for stand-alone use or having the possibility of connecting to an external computer 40 as also shown in FIG. 3. The connection 42 to the computer may be through USB. The connection to an external PC provides for extended functionalities, such as real-time plotting of data, and monitoring of heater temperature.

The LOAC instrument may be based on a standard enclosure from OKW (model A9085165, combi desk case H), modified with holes for fan, on/off button, fuse, power connector, USB-connection and (micro-)SD memory card.

The internal mechanics of the instrument 30 (the cartridge-carrier) comprise of a slot for inserting the cartridge 10, integrating ball-spring plungers for physical feedback to the user, as well as the top and bottom heaters (with thermal probes) used to facilitate and control the LAMP process. The structure may have holes from the top, to accommodate the light sources, e.g. LEDs, and the light detectors, e.g. phototransistors, used for the detection. The internal mechanics may be made from 3 mm thick PMMA plates, laser cut to the desired shapes, and assembled as a layered “sandwich” structure.

The internal mechanical structure may be designed so that the bottom heater is exposed from the bottom-side; to allow rapid cooling by dragging air from vents in the bottom of the enclosure, below the heater, by turning on the fan. This way, cooling to less than 45° C. can be achieved in under 5 minutes. The entire internal mechanics is fastened to the bottom of the enclosure through the bottom plate of the assembly. This bottom plate covers the entire bottom of the enclosure, and further facilitates mounting of the electronics.

The electronics parts may comprise a controlling unit (Arduino Mega); custom-made PCBs comprising an optical sensor board, a sensor handling board, a power handling board, and a user interface (LCD display, LEDs & membrane push-buttons); at least one, but preferably two heaters, each comprising of a resistive heater glued to and 0.5 mm to 5 mm thick aluminum plate, preferably 1 mm thick, and a fan for dragging ambient air through the instrument, in order to achieve faster cooling.

The user interface 32 shown in FIG. 2 comprises membrane electronics (numerical pad, choose options, etc.) combined with a display 34, e.g. a Liquid Crystal Display (LCD) display, for showing instructions and give text feedback to the user. The user interface 32 also comprises a number of LEDs 36 to show status of the system (“Ready”, “Heating” & “Cooling”). The user interface 32 may also comprise LEDs 38 for showing whether the samples being tested are positive (red color) or negative (green color). A signal for uncertain or suspicious results (yellow color) may also be included in the setup. By using membrane electronics, it is possible to combine all user interface elements into one unit, with a nice, user-friendly look and feel. The numbers (1, 2, 3, 4) and letters (A, B, C, D) are used for identifying the individual test well 22.

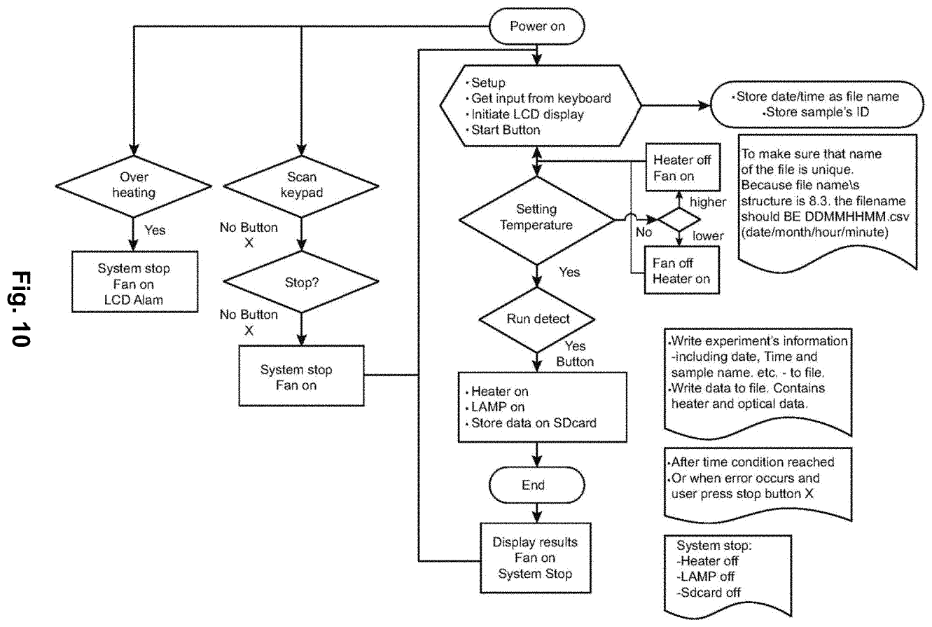

Overall, the LOAC system both works as a stand-alone device, capable of providing a simple positive/negative test answer, and together with a PC based application, for more advanced data representing and handling, such as giving quantitative results (provided a calibration has been performed). FIG. 10 show a flowchart for stand-alone use of the LOAC system is exemplified.

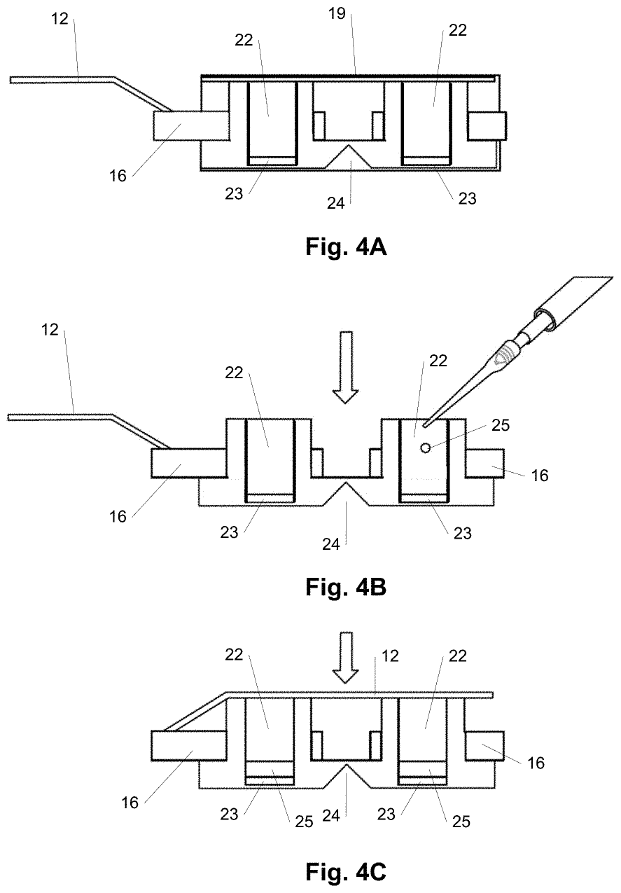

The general protocol for using the LOAC cartridges is shown in FIGS. 4A-C. In its current version, the LOAC system 30 needs approximately 5 minutes to heat up, wherefore the system could advantageously be turned on as the first thing, before handling the cartridge 10. The LOAC cartridge 10 will normally come pre-loaded with the reagents 23 necessary to perform the LAMP (the LAMP kit). To keep the LAMP kit stable and correctly placed in the reaction wells during transport, the reaction mixture is gelified. The LOAC cartridge 10 may come vacuum packed and sealed with a temporary seal. To use the cartridge 10, the temporary seal 19 is first removed, after which the sample 25 to be analyzed is pipetted on top of the gelified LAMP kit. Afterwards, the release liner 13 is removed from the lid 12, and the test wells 22, now loaded with sample 25, is sealed by the user. Care should be taken to assure a good bond of the sealing tape on all wells. Finally, the LOAC cartridge 10 is inserted in the LOAC system 30.

The detection principle is based on light absorption measurements. When detecting using LAMP, light intensity diminishes as the result of a precipitate forming (In LAMP magnesium pyrophosphate) during amplification of DNA. Light from a light source 26 is sent through the sample in the test well 22. A photodetector 28 is used to measure the intensity in real-time. The light source may be an LED, e.g. a Green light (535 nm) LED. The photodetector 28 may be a phototransistor (BPW-77NA), which is used to measure the intensity in real-time, sampling 1 time per second.

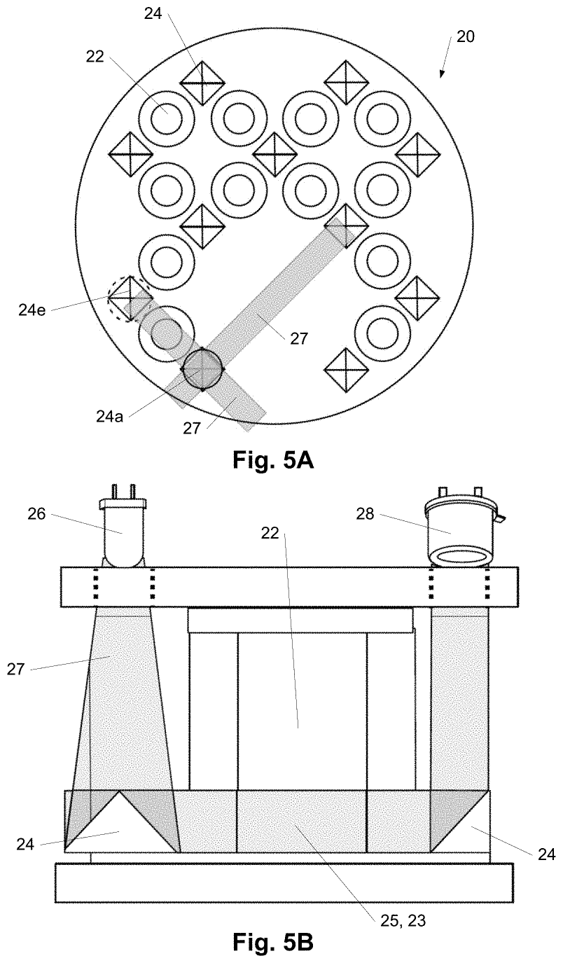

The optical structures 24 embedded directly in the clear polymer of the injection molded part of the LOAC chip 20, deflects the light, coming from the top, in the plane of the chip, in four directions as shown in FIG. 5B. Due to the high amount of DNA made during amplification, to avoid contamination and false positives in subsequent tests, the LOAC cartridge 10 should be disposed of directly, taking care not to compromise the seal of the wells during handling.

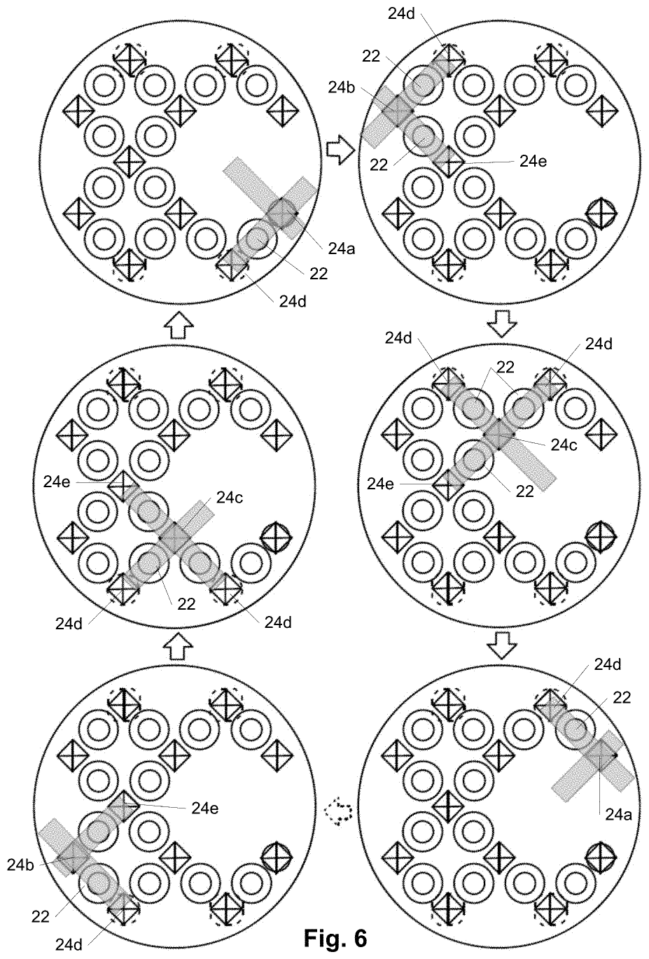

In FIG. 6 an embodiment of the present cartridge 10 comprising a chip 20 with twelve test wells 22, and 11 pyramids 24 are used. The optical detection apparatus 30 comprises six LEDs for illuminating six of the optical pyramids 24 and five phototransistors act as light detectors 28 and are arranged such that by sequentially turning on and off the six LEDs and the five phototransistors, it is possible to sample all twelve test wells 22 individually.

As shown in FIGS. 5A and 6, the LOAC chip 20 comprises optical structures 24 positioned on opposite sides of each of the test wells 22. The optical structures 24 are aligned such that light reflected from one optical structure passes through a test well 22 in at least one of the directions in which the optical structure 24 reflects the light in. Due to the pyramid design, some of the optical structures 24 ensures that several test wells 22 are illuminated by the reflection of the light incident on that optical structure 24. The optical structures marked 24a, 24b, 24c in FIGS. 5A and 6 are examples of optical structures which reflects light through one, two, and three test wells 22, respectively. Likewise, the optical structures marked with 24d and 24e receives light after having been directed through two and four test wells 22, respectively. The pyramid design of the optical structures 24 therefore reduces the number of light sources 26 and photo detectors 28 needed in the apparatus 30 since each optical structure 24 reflects light from a light source 26 in multiple directions, or directs light incident at least two directions after having passed through a multiple of test wells 22 to one photo detector 28.

In FIG. 6, the cycle start is the lowest left-hand image. The arrows mark the sequence.

### Examples

Adaptation and Testing of the LOAC System and Cartridge to Detect Avian Influenza Virus.

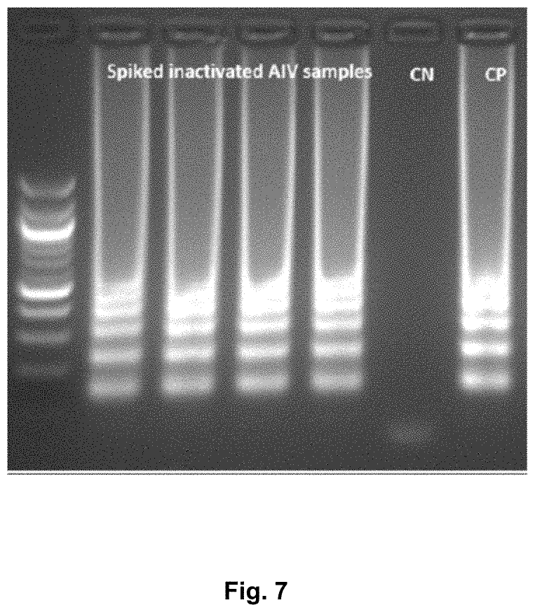

The LAMP kit for detection of AIV (Avian Influenza Virus) was developed. A gelification procedure has been developed for an AIV-LAMP kit. Primary results from testing of the AIV-LAMP kit are herein disclosed. In the reported tests, the LOAC system and cartridge were used to detect AIV in spiked samples. FIG. 7 shows the result from one of the experiments performed to test the performance of AIV LAMP kit, using the LOAC system to detect different AIV collected from Europe and America. Poultry oropharyngeal swab samples were collected and spiked with inactivated AIV virus, before used as samples in the experiment.

In FIG. 7 there is shown a test of one-step RT-LAMP (spiked inactivated AIV in oral swap samples) in a cartridge 10 using 2% agarose gel electrophoresis to confirm the LAMP amplification product. Lane 1: molecular marker; Lane2-5: LAMP amplification products of different AIV spiked samples. Lane 6: Negative control and lane 7: positive control.

In addition, the sensitivity and specificity of the AIV-LAMP kit was tested and determined. First, tests of the sensitivity and specificity of the AIV-LAMP kit were performed on tube, using one-step AIV RT-LAMP kit.

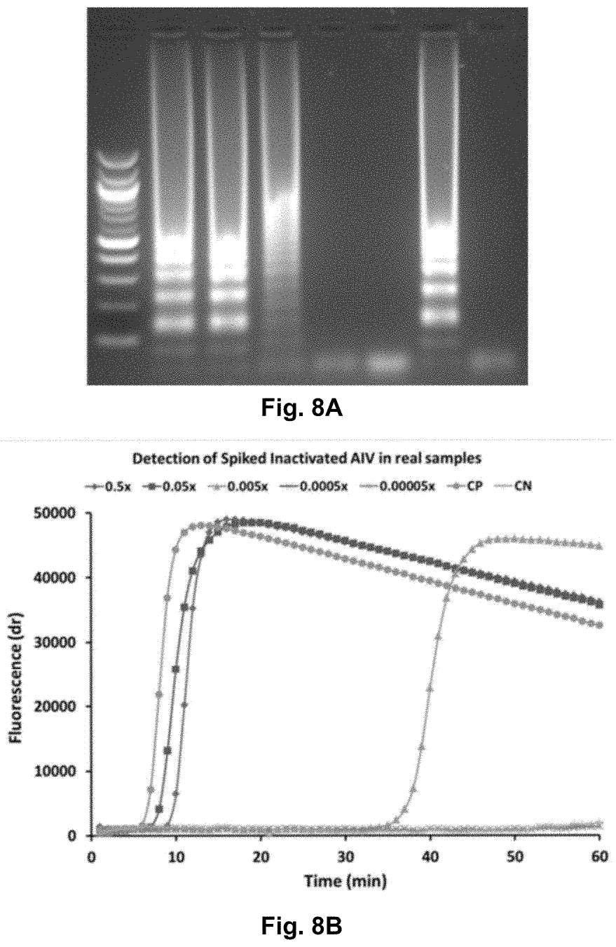

FIGS. 8A-B show the result of one of the sensitivity tests of the one-step RT-LAMP of real samples, using one-step on tube real-time PCR. A serial dilution of inactivated AIV from 0.5-0.000005 inactivated AIV virus were prepared and spiked in a chicken oral swap sample. Afterwards, the samples were tested using the AIV-LAMP kit, showing a limit of detection as low as 0.005 concentration. FIG. 8A: On-tube testing the sensitivity of one-step RT-AIV-LAMP kit, to detect AIV in a serial dilution of AIV, spiked in a poultry oral swab sample. FIG. 8B: Fluorescence curves of one-step RT-AIV-Lamp kit to detect AIV in serial AIV spiked sample. The measurements are conducted using 2% agarose gel electrophoresis to confirm the LAMP amplification product. Lane 1: molecular marker; Lane2-5: LAMP amplification product, Lane 6: Newcastle virus (act as negative control) lane 7: Positive control and Lane 8: Negative control for the AIV-LAMP kit.

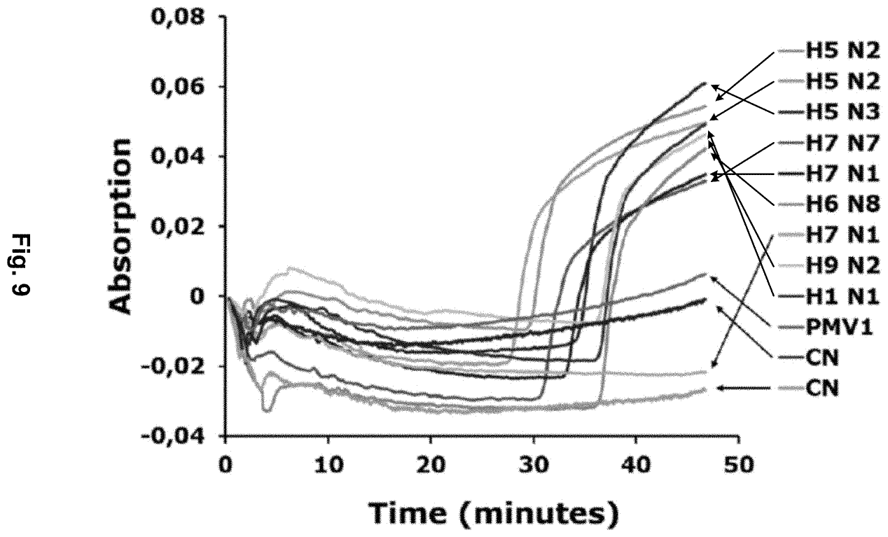

Similar tests of sensitivity and specificity of the AIV-LAMP kit, using the LOAC system and cartridges, has been performed. Primary results showed that of 9 different inactivated AIV virus (with different type), the LOAC system and cartridge can detect AIV from 5 of the 9 inactivated AIV samples. FIG. 9: Detection of AIV using the LOAC system and cartridge to detect inactivated AIV with different subtypes. The plot shows absorption curves of the one-step RT-AIV-Lamp kit used to detect AIV in serial AIV spiked samples.

Adaptation and testing of the LOAC system and cartridge to detect Salmonella spp. A LAMP kit for detection of Salmonella, namely Sal-LAMP kit, with gelified reagents for ready-to-use tests, has been developed (data not shown). The stability of the gelified Sal-LAMP Kit has been tested and determined. The results show that the Sal-LAMP kit is stable for at least 30 weeks at 4° C., and 7 weeks at room temperature.

In addition, The LOAC system and cartridges were used to detect Salmonella spp. in 24 of the 80 samples collected from a poultry slaughter in Denmark. The performance of the LOAC system and cartridges was evaluated and compared with other methods (bacterial culture; Salmonella 12 PCR, LAMP Liquid on tube). Of the 24 samples tested using the LOAC system and cartridges, the results are in 100% agreement with conventional culture method.

Adaptation and Testing of the LOAC System and Cartridge to Detect Campylobacter

A Camp-LAMP kit for detection of both Campylobacter jejuni and Campylobacter coli, the two most common (95%) Campylobacter species found in poultry and human campylobacteriosis, has been developed (data not shown). The Camp-LAMP kit, based on gelified reagents, has been developed and completed. The kit was tested first with campylobacter-spiked sample, before using the kit to detect campylobacter from samples collected from a Danish poultry slaughter.

Use of the LOAC System and Cartridge to Detect SARS-CoV-2

The LOAC system of the invention was successfully used to test for SARS-CoV-2 in accordance with the disclosed objects and methods of the invention and embodiments thereof.

In the below table is shown a master mix, which is used for the detection of SARS-CoV-2:

TABLE 1

| | | No. of | | 1X Final | 1X Final | |
|---|---|---|---|---|---|---|
| LAMP | Stock | reactions | | concentration | concentration | |
| Component | Concentration | 50 | 1X | (μM) | (μM) | |
| F3 100 μM | 100 | 3 | 0.06 | 0.2 | 0.2 | μM |
| B3 100 μM | 100 | 3 | 0.06 | 0.2 | 0.2 | μM |
| FIP 100 μM | 100 | 21 | 0.42 | 1.4 | 1.6 | μM |
| BIP 100 μM | 100 | 21 | 0.42 | 1.4 | 1.6 | μM |
| LF 100 μM | 100 | 12 | 0.24 | 0.8 | 1 | μM |
| LB 100 μM | 100 | 12 | 0.24 | 0.8 | 1 | μM |
| dNTPs 100 mM | 100 | 21 | 0.42 | 1.4 | 1.4 | mM |
| Betaine 5M | 5 | 75 | 1.5 | 0.25 | 0 | mM |
| MgSO4 | 100 | 22.5 | 0.45 | 1.5 | 1.5/2 | μM |
| Buffer 10X | 10 | 150 | 3 | 1 | 1X | |
| H2O | | 1084.5 | 21.69 | | | |
| RTx | | 30 | 0.6 | 9 U/30 μL | 6 U/20 μL | |
| Bst | | 75 | 1.5 | 8 | 12 | U |
| polymerase | | | | | | |
| Total | | 445.5 | 8.91 | | | |

B refers to backward and F refers to forward.

The Mastermix is added to the test well to together with a gelling material to make the test wells ready to receive the test sample.

The below table shows the master mix used:

TABLE 2

| | | 1X final | |
|---|---|---|---|
| Components | Volume (μL) | concentration | X samples |
| 1X Mastermix | 8.91 | 1X | 445.5 |
| 2% (gel and | 15 | 1.00% | 750 |
| trehalose) solution | | | |
| H2O | 2.09 | | 104.5 |
| Total MX | | | 1300 |
| DNA template | 4 | | |
| Total reaction | 30 | | |
| volume | | | |

The sequence of primers F3, B3, FIP, BIP, LF, and LB are given in the below table:

TABLE 3

| SEQ | Primer | |
|---|---|---|
| ID NO | name | Sequence 5′-3′ direction |
| 1 | GeneN-B-F3 | ACCGAAGAGCTACCAGACG |
| 2 | GeneN-B-B3 | TGCAGCATTGTTAGCAGGAT |
| 3 | GeneN-B- | TCTGGCCCAGTTCCTAGGTA |
| | FIP | GTTCGTGGTGGTGACGGTAA |
| 4 | GeneN-B- | AGACGGCATCATATGGGTTG |
| | BIP | CACGGGTGCCAATGTGATCT |
| 5 | GeneN-B-LF | CCATCTTGGACTGAGATCTT |
| | | TCATT |
| 6 | GeneN-B-LB | ACTGAGGGAGCCTTGAATACA |

The invention is further described in the following non-limiting embodiments.

1. A kit for amplifying a SARS-CoV-2 virus nucleic acid comprising a first and a second primer, wherein the first and second primer has at least 80% homology to the nucleic acid sequences of any one of the following primer sets: SEQ ID NO 1 and SEQ ID NO 2, respectively or SEQ ID NO 3 and SEQ ID NO 4 respectively or SEQ ID NO 5 and SEQ ID NO 6 respectively.

2. A kit for amplifying a SARS-CoV-2 virus nucleic acid comprising a first primer comprising the nucleic acid sequence of SEQ ID NO 1 and a second primer comprising the nucleic acid sequence of SEQ ID NO 2.

3. A kit for amplifying a SARS-CoV-2 virus nucleic acid comprising a first primer comprising the nucleic acid sequence of SEQ ID NO 3 and a second primer comprising the nucleic acid sequence of SEQ ID NO 4.

4. A kit for amplifying a SARS-CoV-2 virus nucleic acid comprising a first primer comprising the nucleic acid sequence of SEQ ID NO 5 and a second primer comprising the nucleic acid sequence of SEQ ID NO 6.

5. A kit according to any of embodiments 1-4, further comprising reagents and enzymes for carrying out a PCR reaction.

7. A kit according to any of embodiments 1-5, further comprising reagents and enzymes for carrying out a reverse transcriptase PCR reaction.

8. A kit according to any of embodiments 1-7, further comprising a standard nucleic acid as a qualitative and/or quantitative control for monitoring RNA extraction and/or amplification reaction.

9. Use of a combination of a first primer and a second primer, having at least 80% homology to the nucleic acid sequences of SEQ ID NO 1 and SEQ ID NO 2, or to SEQ ID NO 3 and SEQ ID NO 4 or to SEQ ID NO 5 and SEQ ID NO 6, respectively, for amplification of a COVID19 virus nucleic acid.

10. Use of a combination of a first primer comprising the nucleic acid sequence of SEQ ID NO 1 and a second primer comprising the nucleic acid sequence of SEQ ID NO 2, or a first primer comprising the nucleic acid sequence of SEQ ID NO 3 and a second primer comprising the nucleic acid sequence of SEQ ID NO 4, or a first primer comprising the nucleic acid sequence of SEQ ID NO 5 and a second primer comprising the nucleic acid sequence of SEQ ID NO 6 for amplification of a COVID19 virus nucleic acid.

11. Method for detecting a SARS-CoV-2 virus in a sample comprising the steps of i) performing a RT (reverse transcriptase) polymerase chain reaction (PCR) ii) performing a PCR reaction, using as a first primer a primer having at least 80% homology to SEQ ID NO 1 and a second primer having at least 80% homology to SEQ ID NO 2 iii) detecting the amplified nucleic acid sequence of step ii) wherein steps i)-iii) can be performed sequentially or steps i)-ii) or steps i)-iii) or steps ii)-iii) can be performed simultaneously.

12. Method according to embodiment 11, wherein the first and second primer comprises the nucleic acid sequences of SEQ ID NO 1 and SEQ ID NO 2, respectively.

13. Method for detecting a SARS-CoV-2 virus in a sample comprising the steps of i) performing a RT (reverse transcriptase) polymerase chain reaction (PCR) ii) performing a PCR reaction, using as a first primer a primer having at least 80% homology to SEQ ID NO 3 and a second primer having at least 80% homology to SEQ ID NO 4 iii) detecting the amplified nucleic acid sequence of step ii) wherein steps i)-iii) can be performed sequentially or steps i)-ii) or steps i)-iii) or steps ii)-iii) can be performed simultaneously.

14. Method according to embodiment 13, wherein the first and second primer comprises the nucleic acid sequences of SEQ ID NO 3 and SEQ ID NO 4, respectively.

15. Method for detecting a SARS-CoV-2 virus in a sample comprising the steps of i) performing a RT (reverse transcriptase) polymerase chain reaction (PCR) ii) performing a PCR reaction, using as a first primer a primer having at least 80% homology to SEQ ID NO 5 and a second primer having at least 80% homology to SEQ ID NO 6 iii) detecting the amplified nucleic acid sequence of step ii) wherein steps i)-iii) can be performed sequentially or steps i)-ii) or steps i)-iii) or steps ii)-iii) can be performed simultaneously.

16. Method according to embodiment 15, wherein the first and second primer comprises the nucleic acid sequences of SEQ ID NO 5 and SEQ ID NO 6, respectively.

17. Method according to any of embodiments 11-16, which further comprises the steps of amplifying and detecting a standard nucleic acid.

18. Method according to any of embodiments 11-17, wherein step ii) and iii) are performed as a real-time PCR reaction.

19. A primer for detecting a SARS-CoV-2 virus, wherein said primer comprises at least 80% homology to the nucleic acid sequence of SEQ ID NO 1.

20. A primer for detecting a SARS-CoV-2 virus, wherein said primer comprises the nucleic acid sequence of SEQ ID NO 1.

21. A primer for detecting a SARS-CoV-2 virus, wherein said primer comprises at least 80% homology to the nucleic acid sequence of SEQ ID NO 2.

22. A primer for detecting a SARS-CoV-2 virus, wherein said primer comprises the nucleic acid sequence of SEQ ID NO 2.

23. A primer for detecting a SARS-CoV-2 virus, wherein said primer comprises at least 80% homology to the nucleic acid sequence of SEQ ID NO 3.

24. A primer for detecting a SARS-CoV-2 virus, wherein said primer comprises the nucleic acid sequence of SEQ ID NO 3.

25. A primer for detecting a SARS-CoV-2 virus, wherein said primer comprises at least 80% homology to the nucleic acid sequence of SEQ ID NO 4.

26. A primer for detecting a SARS-CoV-2 virus, wherein said primer comprises the nucleic acid sequence of SEQ ID NO 4.

27. A primer for detecting a SARS-CoV-2 virus, wherein said primer comprises at least 80% homology to the nucleic acid sequence of SEQ ID NO 5.

28. A primer for detecting a SARS-CoV-2 virus, wherein said primer comprises the nucleic acid sequence of SEQ ID NO 5.

29. A primer for detecting a SARS-CoV-2 virus, wherein said primer comprises at least 80% homology to the nucleic acid sequence of SEQ ID NO 6.

30. A primer for detecting a SARS-CoV-2 virus, wherein said primer comprises the nucleic acid sequence of SEQ ID NO 6.

31. A composition comprising six primers, one according to each of embodiments 19 or 20 and 21 or 22 and 23 or 24.

32. A composition comprising the primers of embodiment 31, further comprising dNTPs, Betaine, MgSO4, a buffer, RTX and a DNA polymerase.

The invention is further described in the following non-limiting items.

1. A portable optical detection cartridge configured for being received in an optical detection apparatus, wherein the portable optical detection cartridge comprises:
a plurality of test wells, wherein a number of the plurality of test wells are pre-loaded with suitable reagents allowing a loop-mediated isothermal amplification (LAMP) between a test sample and the reagents to take place within each test well when the test sample is administered to the test well;
a plurality of optical structures, wherein each optical structure is configured for reflecting light in at least three reflection directions.

2. The portable optical detection cartridge according to any preceding item, wherein each optical structure is configured for reflecting light in four reflection directions.

3. The portable optical detection cartridge according to any preceding item, wherein each optical structure is shaped as a pyramid having four reflecting surfaces.

4. The portable optical detection cartridge according to any preceding item, wherein each optical structure is shaped as a pyramid having four reflecting surfaces and each optical structure is configured for reflecting light in four reflection directions.

5. The portable optical detection cartridge according to any preceding item, wherein each optical structure is a total internal reflection structure.

6. The portable optical detection cartridge according to any preceding item, wherein each optical structure is configured for receiving light from the optical detection apparatus and/or configured for transmitting light from another optical structure and/or light emitted from the test sample to the optical detection apparatus.

7. The portable optical detection cartridge according to any preceding item, wherein at least one of the optical structures is configured for receiving light from the optical detection apparatus and for transmitting the light to the test sample and at least one other of the optical structures is configured to transmit light emitted from the test sample or passing through the test sample to the optical detection apparatus.

8. The portable optical detection cartridge according to any preceding item, wherein the portable optical detecting cartridge is configured for being received in an optical detection apparatus comprises six light sources and five light detectors and wherein the portable optical detection cartridge comprises eleven optical structures, wherein each of the optical structures are configured for either reflecting light from one of the six light sources or reflecting light to one of the five light detectors when the portable optical detection cartridge is inserted into the optical detection apparatus.

9. The portable optical detection cartridge according to any preceding item, wherein along at least two of the reflection directions, defined by each optical structure, another optical structure is positioned and wherein two optical structures are positioned on opposite sides of each test well.

10. The portable optical detection cartridge according to any preceding item, wherein the suitable reagents are gelified and wherein the suitable reagents form a sample mixture when added the test sample, such that the sample mixture is configured for emitting fluorescence light when illuminated with light from the optical detection apparatus, wherein a first of the plurality of optical structures are configured for reflecting the light from the optical detection apparatus through a test well comprising the sample mixture and at least a second of the plurality of optical structures are configured for reflecting the emitted fluorescence to the optical detection apparatus.

11. The portable optical detection cartridge according to any preceding item, wherein the optical detection apparatus comprises six light sources and five light detectors and wherein the portable optical detection cartridge comprises eleven optical structures, wherein each of the optical structures are configured for reflecting light from one of the six light sources or to one of the five light detectors when the portable optical detection cartridge is inserted into the optical detection apparatus.

12. The portable optical detection cartridge according to any preceding item, wherein the plurality of test wells is twelve test wells, and where the plurality of optical structures is eleven optical structures.

13. The portable optical detection cartridge according to any preceding item, wherein along at least two of the reflection directions, defined by each optical structure, another optical structure is positioned.

14. The portable optical detection cartridge according to any preceding item, wherein at least one of the optical structures is configured for reflecting light to and/or from four of the other optical structures.

15. The portable optical detection cartridge according to any preceding item, wherein each optical structure is configured for reflecting light at a reflection angle between 45-135 degrees, such as between 60-120 degrees, such as 75-105 degrees, such as 85-95 degrees, or such as approximately 90 degrees.

16. The portable optical detection cartridge according to any preceding item, wherein two optical structures are positioned on opposite sides of each test well.

17. The portable optical detection cartridge according to any preceding item, wherein each optical structure is configured for reflecting light in five, six, seven, eight, or more reflection directions.

18. The portable optical detection cartridge according to any preceding item, wherein the portable optical detection cartridge comprises a chip comprising the plurality of test wells and the plurality of optical structures, a holder for holding the chip, and a lid for closing access to the test wells after sample has been supplied to one or more of the test wells.

19. The portable optical detection cartridge according to any preceding claim, wherein the portable optical detection cartridge further comprises a temporary seal for closing access to the test wells, wherein the temporary seal is removed before test sample is supplied to one or more of the test wells.

20. The portable optical detection cartridge according to item 18, wherein the chip is an injection molded chip.

21. The portable optical detection cartridge according to item 18 or 20, wherein the holder is fabricated from a black polymeric material, and the lid is made from polymerase chain reaction (PCR) tape.

22. The portable optical detection cartridge according to any preceding item, wherein the plurality of test wells are absent of fluidic-connection channels connecting the test wells.

23. The portable optical detection cartridge according to any preceding item, wherein the plurality of test wells comprises at least two control test wells including at least one negative control test well and at least one positive control test well.

24. The portable optical detection cartridge according to any preceding item, wherein the suitable reagents are gelified to ensure that they remain inside the test well during handling prior to supplying the test sample to the plurality of test wells.

25. The portable optical detection cartridge according to any preceding item, wherein the sample and the suitable reagents form a sample mixture configured for emitting fluorescence light when illuminated with light from the optical detection apparatus, wherein a first of the plurality of optical structures are configured for reflecting the light from the optical detection apparatus through a test well with the sample mixture and at least a second of the plurality of optical structures are configured for reflecting the emitted fluorescence to the optical detection apparatus.

26. The portable optical detection cartridge according to any preceding item, wherein the plurality of test wells are pre-loaded with reagents selective for a disease pathogen, such as adenoviruses, herpesviruses, poxviruses, parvoviruses, reoviruses, coronaviruses, picornaviruses, togaviruses, orthomyxoviruses, rhabdoviruses, retroviruses, or hepadnaviruses.

27. The portable optical detection cartridge according to any preceding item, wherein the plurality of test wells are pre-loaded with reagents selective for a disease pathogen such as severe acute respiratory syndrome coronavirus 2 (SARS-CoV-2).

28. A lab-on-a-chip system comprising the portable optical detection cartridge according to any of the items 1-27 and an optical detection apparatus configured for receiving the portable optical detection cartridge, wherein the portable optical detection cartridge comprises at least a first optical structure and a second optical structure, and wherein the optical detection apparatus comprises at least one light source for illuminating the first optical structure and at least one light detector for receiving light transmitted from the second optical structure.

29. The lab-on-a-chip system according to item 28, wherein the optical detection apparatus comprises a plurality of light sources and a plurality of light detectors.

30. The lab-on-a-chip system according to any of the items 28-29, wherein the light source(s) are light emitting diodes (LEDs).

31. The lab-on-a-chip system according to item 28, wherein the optical detection apparatus comprises a plurality of light sources and a plurality of light detectors and wherein the light sources are light emitting diodes (LEDs).

32. The lab-on-a-chip system according to any of the items 28-31, wherein the optical detection apparatus is configured for performing a detection cycle comprising a sequence of illumination-detection steps, wherein in each illumination-detection step:
a light source illuminates a first optical structure;
one or more test wells are illuminated by the light reflected by the optical structure through the one or more test wells; and
one or more second optical structures reflect light emitted from the sample in the test well and/or the light transmitted through the test well to one or more light detectors in the optical detection apparatus.

33. The lab-on-a-chip system according to item 32, wherein only one light source is turned on in each illumination-detection step.

34. The lab-on-a-chip system according to any of the items 32-33, wherein each light detector is configured for detecting light transmitted through and/or emitted from at least two test wells.

35. The lab-on-a-chip system according to item 34, wherein each light detector is configured for detecting light transmitted through at least two test wells.

36. The lab-on-a-chip system according to item 34, wherein each light detector is configured for detecting light emitted from at least two test wells.

37. The lab-on-a-chip system according to any of the items 28-36, wherein the optical detection apparatus comprises six light sources and five light detectors and wherein the portable optical detection cartridge comprises eleven optical structures each reflecting light from one of the six light sources or to one of the five light detectors.

38. The lab-on-a-chip system according to item 37, wherein the detection cycle comprises six illumination-detection steps.

39. The lab-on-a-chip system according to any of the items 28-38, wherein the optical detection apparatus comprises one or more heating sources for heating the test wells.

40. The lab-on-a-chip system according to any of the items 28-39, wherein the optical detection apparatus is a loop-mediated isothermal amplification (LAMP) optical detection apparatus.

41. The lab-on-a-chip system according to any of the items 39-40, wherein the heating source(s) are configured for maintaining the temperature approximately constant during detection.

42. The lab-on-a-chip system according to any of the items 28-41, wherein the optical detection apparatus comprises six light sources and five light detectors, wherein the portable optical detection cartridge comprises eleven optical structures, wherein each of the optical structures are configured for either reflecting light from one of the six light sources or reflecting light to one of the five light detectors, wherein the detection cycle comprises six illumination-detection steps each step turning on a different of the six light sources, and wherein the optical detection apparatus comprises one or more heating sources for heating the test wells, wherein the one or more heating sources are configured for maintaining an approximately constant temperature during the loop-mediated isothermal amplification between the test sample and the reagents and for maintaining an approximately constant temperature during detection.

43. Use of the portable optical detection cartridge according to any of the items 1-27 or the lab-on-a-chip system according to any of the items 28-42 for qualitative or quantitative detection of a disease pathogen in a test sample.

44. A method of detecting a presence of a disease pathogen in a test sample, wherein the method comprises the steps of:
Adding the test sample to at least one test well in the portable optical detection cartridge according to any of the items 1-27;
Inserting the portable optical detection cartridge into a optical detection apparatus;
Heating the portable optical detection cartridge within the optical detection apparatus to a predetermined temperature, preferably between 60 and 70 C;
Maintaining the predetermined temperature until the method of detecting the disease pathogen in the test sample is complete;
Leaving the loop-mediated isothermal amplification between the test sample and the reagents to take place within the test well for a predetermined amount of time;
Emitting light from a light source in the optical detection apparatus to at least one of the optical structures hereby emitting light to the at least one test well in the portable optical detection cartridge;
Detecting light with a photo detector in the optical detection apparatus, wherein the light is emitted from the at least one test well in the portable optical detection cartridge to one of the optical structures reflecting light to the photodetector, hereby detecting the presence of the disease pathogen in the test sample.

### REFERENCES

10 lab-on-a-chip cartridge
12 lid
13 release liner
14 tape
16 holder
19 temporary seal
20 chip
22 test well
23 gel/reagents
24 optical structure
24a, 24b, 24c optical structure receiving light from a light source
24d, 24e optical structure directing light to the detector
25 test sample
26 light source, e.g. an LED
27 light
28 light detector, e.g. a photodetector
30 optical detection apparatus/instrument
32 user interface
34 display
36 LED
38 LED
40 external computer
42 connection between the computer and the optical detection apparatus

1. A portable optical detection cartridge configured for being received in an optical detection apparatus, wherein the portable optical detection cartridge comprises:
a lid;
a holder; and
a chip;
wherein the chip at least comprises:
a plurality of test wells, wherein a number of the plurality of test wells are pre-loaded with suitable reagents allowing a loop-mediated isothermal amplification (LAMP) between a test sample and the reagents to take place within each test well when the test sample is administered to the test well; and
a plurality of optical structures, wherein each optical structure is configured for reflecting light in a 90 degree angle in at least three reflection directions;
wherein at least one of the optical structures is configured for receiving light from the optical detection apparatus and for transmitting the light to the test sample; and
wherein two optical structures are positioned on opposite sides of each test well.

2. The portable optical detection cartridge according to claim 1, wherein each optical structure is shaped as a pyramid having four reflecting surfaces and each optical structure is configured for reflecting light in four reflection directions.

3. The portable optical detection cartridge according claim 1, wherein each optical structure is a total internal reflection structure.

4. The portable optical detection cartridge according to claim 1, wherein at least one of the optical structures is configured for receiving light from the optical detection apparatus and for transmitting the light to the test sample and at least one other of the optical structures is configured to transmit light emitted from the test sample or passing through the test sample to the optical detection apparatus.

5. The portable optical detection cartridge according to claim 1, wherein the portable optical detecting cartridge is configured for being received in an optical detection apparatus comprises six light sources and five light detectors and wherein the portable optical detection cartridge comprises eleven optical structures, wherein each of the optical structures are configured for either reflecting light from one of the six light sources or reflecting light to one of the five light detectors when the portable optical detection cartridge is inserted into the optical detection apparatus.

6. The portable optical detection cartridge according to claim 1, wherein along at least two of the reflection directions, defined by each optical structure, another optical structure is positioned and wherein two optical structures are positioned on opposite sides of each test well.

7. The portable optical detection cartridge according to claim 1, wherein the suitable reagents are gelified and wherein the suitable reagents form a sample mixture when added the test sample, such that the sample mixture is configured for emitting fluorescence light when illuminated with light from the optical detection apparatus, wherein a first of the plurality of optical structures are configured for reflecting the light from the optical detection apparatus through a test well comprising the sample mixture and at least a second of the plurality of optical structures are configured for reflecting the emitted fluorescence to the optical detection apparatus.

8. A lab-on-a-chip system comprising the portable optical detection cartridge according to claim 1 and an optical detection apparatus configured for receiving the portable optical detection cartridge, wherein the portable optical detection cartridge comprises at least a first optical structure and a second optical structure, and wherein the optical detection apparatus comprises at least one light source for illuminating the first optical structure and at least one light detector for receiving light transmitted from the second optical structure.

9. The lab-on-a-chip system according to claim 8, wherein the optical detection apparatus comprises a plurality of light sources and a plurality of light detectors and wherein the light sources are light emitting diodes (LEDs).

10. The lab-on-a-chip system according to claim 8, wherein the optical detection apparatus is configured for performing a detection cycle comprising a sequence of illumination-detection steps, wherein in each illumination-detection step:
a light source illuminates a first optical structure;
one or more test wells are illuminated by the light reflected by the optical structure through the one or more test wells; and
one or more second optical structures reflect light emitted from the sample in the test well and/or the light transmitted through the test well to one or more light detectors in the optical detection apparatus.

11. The lab-on-a-chip system according to claim 10, wherein only one light source is turned on in each illumination-detection step.

12. The lab-on-a-chip system according to claim 10, wherein each light detector is configured for detecting light transmitted through and/or emitted from at least two test wells.

13. The lab-on-a-chip system according to claim 8, wherein the optical detection apparatus comprises six light sources and five light detectors, wherein the portable optical detection cartridge comprises eleven optical structures, wherein each of the optical structures are configured for either reflecting light from one of the six light sources or reflecting light to one of the five light detectors, wherein the detection cycle comprises six illumination-detection steps each step turning on a different of the six light sources, and wherein the optical detection apparatus comprises one or more heating sources for heating the test wells, wherein the one or more heating sources are configured for maintaining an approximately constant temperature during the loop-mediated isothermal amplification between the test sample and the reagents and for maintaining an approximately constant temperature during detection.

14. A method for qualitative or quantitative detection of a disease pathogen in a test sample comprising providing the lab-on-a-chip system according to claim 8.

15. A method of detecting a presence of a disease pathogen in a test sample, wherein the method comprises the steps of:
Adding the test sample to at least one test well in the portable optical detection cartridge according to claim 1;
Inserting the portable optical detection cartridge into a optical detection apparatus;
Heating the portable optical detection cartridge within the optical detection apparatus to a predetermined temperature, preferably between 60° C. and 70° C.;
Maintaining the predetermined temperature until the method of detecting the disease pathogen in the test sample is complete;
Leaving the loop-mediated isothermal amplification between the test sample and the reagents to take place within the test well for a predetermined amount of time;
Emitting light from a light source in the optical detection apparatus to at least one of the optical structures hereby emitting light to the at least one test well in the portable optical detection cartridge;
Detecting light with a photo detector in the optical detection apparatus, wherein the light is emitted from the at least one test well in the portable optical detection cartridge to one of the optical structures reflecting light to the photodetector, hereby detecting the presence of the disease pathogen in the test sample.
